(19) United States
(12) Patent Application Publication
(10) Pub. No.: US 2026/0191430 A1
(43) Pub. Date: Jul. 9, 2026

(54) HUMAN BODY MOTION GAIT ANALYSIS USING LIDAR-BASED 3D GAIT MEASUREMENTS

(71) Applicant: Carecam Pte. Ltd., Singapore (SG)

(72) Inventors: Ramanpreet Singh PAHWA, Ang Mo Kio (SG); Ching Kiat Elson YONG, Singapore (SG); Ling GUO, Singapore (SG)

(73) Assignee: Carecam Pte. Ltd., Singapore (SG)

(21) Appl. No.: 19/013,776

(22) Filed: Jan. 8, 2025

(51) Int. Cl. A61B 5/11

(52) U.S. Cl. CPC A61B 5/112; A61B 5/1128

(57) ABSTRACT

In certain implementations a computer-implemented method, computer program product and systems for performing gait analysis using lidar based three dimensional measurements is provided. Embodiments of the present invention can identify at least one object from received video information. Embodiments of the present invention can then estimate points on the object to depict two dimensional and three dimensional poses of the object based on a threshold number of frames depicting the object. Based on the estimated three dimensional poses, embodiments of the present invention can then generate measurements for biomechanical parameters that characterize movement of the object based on the estimated points on the object.

### TECHNICAL FIELD

The present disclosure relates generally to gait analysis, and in particular, to lidar based three dimensional gait measurements.

### BACKGROUND

Lidar, or light detection and ranging, is a remote sensing technology that uses laser pulses to measure distances and generate 3D models of objects and environments. Lidar has been widely used in various fields, such as geography, archaeology, forestry, and autonomous driving. Recently, lidar has also been applied to human motion analysis, especially gait analysis, which is the study of human walking patterns and biomechanics.

Gait analysis is important for many applications, such as clinical diagnosis, rehabilitation, sports performance, and human-computer interaction. However, traditional methods of gait analysis, such as optical motion capture, inertial sensors, and pressure sensors, have some limitations, such as high cost, complex setup, occlusion, calibration, and privacy issues. Lidar, on the other hand, offers some advantages, such as low cost, easy deployment, robustness to lighting and movement drift, larger coverage, full body tracking, and preservation of anonymity.

Lidar sensors are devices that emit and receive laser pulses to measure the distance and reflectance of objects in the scene. The basic principle of Lidar is to calculate the time-of-flight (TOF) of the laser pulse, which is the time difference between the emission and reception of the pulse. By multiplying the TOF by the speed of light, the distance between the sensor and the object can be obtained. By scanning the laser beam across the scene, a 3D point cloud of the scene can be generated, which is a collection of points with coordinates and reflectance values.

### LIDAR SENSORS

There are different types of Lidar sensors, depending on the scanning mechanism, the wavelength, and the number of beams. The scanning mechanism determines how the laser beam is directed and moved across the scene. There are two main types of scanning mechanisms: mechanical and solid-state. Mechanical Lidar sensors use rotating mirrors or prisms to steer the laser beam, while solid-state Lidar sensors use electronic or optical components, such as microelectromechanical systems (MEMS), liquid crystal, or optical phased arrays, to modulate the beam direction. Mechanical Lidar sensors typically have higher resolution and range, but lower speed and reliability, than solid-state Lidar sensors.

The wavelength of the laser beam affects the penetration and reflectance of the objects in the scene. There are two main wavelengths used in Lidar sensors: near-infrared (NIR) and visible. NIR Lidar sensors use lasers with wavelengths between 700 and 1100 nm, which are invisible to the human eye and can penetrate through fog and smoke [8]. Visible Lidar sensors use lasers with wavelengths between 400 and 700 nm, which are visible to the human eye and can provide color information [9]. NIR Lidar sensors are more common and suitable for outdoor environments, while visible Lidar sensors are more suitable for indoor environments.

The number of beams determines how many laser pulses are emitted and received simultaneously by the sensor. There are two main types of Lidar sensors based on the number of beams: single-beam and multi-beam. Single-beam Lidar sensors use one laser source and one detector, and scan the scene sequentially. Multi-beam Lidar sensors use multiple laser sources and detectors, and scan the scene in parallel. Multi-beam Lidar sensors can achieve higher resolution and speed, but also higher cost and complexity, than single-beam Lidar sensors.

### GAIT FEATURE EXTRACTION AND RECOGNITION

Gait feature extraction and recognition are the processes of extracting and identifying meaningful information from the Lidar data related to human gait. Gait features can be classified into two categories: spatial and temporal. Spatial features describe the shape and size of the human body and limbs, such as height, width, length, and angle. Temporal features describe the dynamic changes of the human body and limbs over time, such as speed, frequency, phase, and amplitude. Gait recognition is the task of identifying or verifying the identity of a person based on their gait features.

There are different methods of gait feature extraction and recognition from Lidar data, depending on the level of abstraction, the type of representation, and the type of classifier. The level of abstraction refers to how much detail is preserved or discarded from the raw Lidar data. There are three main levels of abstraction: point cloud, silhouette, and skeleton. Point cloud is the most raw and detailed representation, which consists of the 3D coordinates and reflectance values of each point in the scene. Silhouette is a simplified and projected representation, which consists of the 2D binary image of the human body outline. Skeleton is a further simplified and abstracted representation, which consists of the 3D coordinates and connections of the human body joints.

The type of representation refers to how the gait features are encoded and stored from the Lidar data. There are two main types of representation: model-based and model-free. Model-based representation assumes a predefined model or structure of the human body and gait, and fits the Lidar data to the model parameters. Model-free representation does not assume any prior model or structure, and extracts the gait features directly from the Lidar data. Model-based representation can achieve higher accuracy and robustness, but also higher complexity and computation, than model-free representation.

Lidar-based gait analysis has various applications and use cases in different domains, such as healthcare, security, entertainment, and education. For example, Lidar can be used to monitor and evaluate the gait of patients with neurological or musculoskeletal disorders, such as stroke, Parkinson's disease, or osteoarthritis. Lidar can provide objective and quantitative measurements of the gait parameters, such as stride length, step width, cadence, and symmetry, and detect abnormal or pathological gait patterns, such as limping, shuffling, or freezing. Lidar can also be used to assess the effectiveness of interventions, such as medication, surgery, or physiotherapy, on the gait recovery and improvement of the patients.

In entertainment, lidar can be used to capture and animate the gait of human actors or performers, such as dancers, athletes, or celebrities, and transfer their motion to virtual or augmented reality environments, such as games, movies, or social media. Lidar can provide a realistic and immersive experience, which can enhance the interaction and engagement of the users, and create new forms of expression and creativity. Lidar can also be used to generate and synthesize novel and diverse gait styles, such as funny, elegant, or scary, and apply them to different characters or scenarios.

### SUMMARY

The invention relates to computer-implemented methods, computer program products, and systems for performing gait analysis using lidar based three dimensional measurements. More particularly the computer-implemented methods, computer program products, and systems receive video information and responsive to receiving video information, identify at least one object depicted in the received video information. In this embodiment, the computer-implemented methods computer program products, and systems estimate points on the object to depict two dimensional and three dimensional poses of the object based on a threshold number of frames depicting the object and generate measurements for biomechanical parameters that characterize movement of the object based on the estimated points on the object.

In an implementation, the computer-implemented method can responsive to detecting more than one object depicted in the received video information, select an object of interest based on camera orientation. The computer-implemented method can further generate bounding boxes corresponding to the object and a portion of the object with corresponding [x, y] coordinate values associated with a center of the bounding boxes and corresponding [w, h] dimensions of the bounding boxes for the object and the portion of the object.

The computer-implemented method can further resize, a region corresponding to the generated bounded box of the object, detect twenty eight two dimensional key points of the object depicted within the bounding boxes for each frame depicted in the received video information, and generate normalized values for the detected twenty eight two dimensional key points. In an embodiment, the computer-implemented method can further identify temporal boundaries for segments of movement based on the generated normalized values for the detected twenty eight two dimensional key points and parameters for frame rate and frame dimension.

The computer-implemented method can align the normalized twenty eight two dimensional key points to a threshold number of frames of a video in a sequential order and estimate three dimensional coordinate locations for the object based on neighboring frame three dimensional pose information in the threshold number of frames. From there, the computer-implemented method can generate normalized three dimensional locations of the twenty eight two dimensional key points.

The computer-implemented method can utilize the three dimensional locations of the twenty eight, two dimensional key points to map and scale of a geometrical integration module, the estimated three dimensional pose into real world coordinates by extracting geometrical information available in lidar sensor data. In this embodiment, the computer-implemented extracts the geometrical information available in lidar sensor data by smoothing the normalized three dimensional locations of the twenty eight two dimensional key points, utilizing lidar data to estimate ground plane equation of a surface the object depicting movement traverses and scaling three dimensional key points for each frame based on object specific measurements to provide real world coordinates for the twenty eight key points in 3D, and performing translation on the scaled three dimensional key points per frame to move the corresponding three dimensional key points to a world coordinate system per frame. The computer-implemented method can further identify frames marking commencement and conclusion of movement depicted by the object and estimate a mean and standard deviation for a distance traveled within one gait cycle. The computer-implemented method can then compute angular measurements for each frame depicted in the received video information.

### BRIEF DESCRIPTION OF THE DRAWINGS

FIG. 1 is a functional block diagram depicting a computing environment for gait estimation using LIDAR-based 3D gait measurements, in accordance with at least one embodiment of the present invention;

FIG. 2 is block diagram of a gait analyzer, in accordance with an embodiment of the present invention;

FIG. 3 is a functional block diagram of certain components of a computing environment for gait estimation using LIDAR-based 3d gait measurements, in accordance with at least one embodiment of the present invention;

FIG. 4 is a functional block diagram depicting an approach for gait estimation, in accordance with at least one embodiment of the present invention;

FIG. 5 is an example workflow for generating gait biomarkers, in accordance with at least one embodiment of the present invention;

FIG. 6 is a flowchart depicting operational steps of a program on a computer within the computing environment of FIG. 1 for analyzing received input, in accordance with an embodiment of the present invention;

FIG. 7 is a flowchart depicting operational steps for ground plane estimation, in accordance with an embodiment of the present invention; and

FIG. 8 is a flowchart depicting operational steps for scaling and translation, in accordance with an embodiment of the present invention.

### DETAILED DESCRIPTION

Embodiments of the present invention recognize that lidar-based gait analysis is a promising and emerging field but also recognize that existing lidar-based gait analysis systems have challenges and limitations. For example, existing systems face problems with processing data. For example, lidar data is sparse, noisy, and incomplete, which can affect the quality and accuracy of the gait feature extraction and recognition. Lidar data is also heterogeneous and diverse, which can vary depending on the type, configuration, and location of the lidar sensor, and the environment, condition, and behavior of the human subject. Therefore, embodiments of the present invention recognize there is a need for more standardized and consistent lidar data collection and processing methods, and more large-scale and comprehensive lidar data sets and benchmarks.

Furthermore, embodiments of the present invention recognize that lidar-based gait feature extraction and recognition methods are complex and computationally intensive, which can limit their scalability and applicability. Typical lidar-based gait feature extraction and recognition methods are also domain-specific and task-dependent, which can reduce their generalizability and adaptability. Therefore, embodiments of the present invention recognize there is a need for more efficient and robust Lidar-based gait feature extraction and recognition methods, and more cross-domain and multi-task Lidar-based gait feature extraction and recognition methods.

Embodiments of the present invention also recognize that lidar-based gait analysis applications are still in their infancy and exploratory stages, which can lack validation and evaluation. Lidar-based gait analysis applications are also subject to ethical and social issues, such as privacy, consent, and bias, which can raise concerns and objections. Therefore, embodiments of the present invention recognize that there is a need for more rigorous and systematic lidar-based gait analysis application development and testing, and more ethical and responsible lidar-based gait analysis application design and deployment.

Traditionally, quantitative gait analysis has relied on marker-based motion capture (MoCap) systems, which uses reflective markers attached to the body and multiple high-speed cameras to track movement. These systems capture marker positions in 3 dimensions with millimeter accuracy enabling precise computation of joint kinematics and spatiotemporal gait parameters. However, these systems are expensive, cumbersome and require significant time, effort and expertise for meticulous marker placement and data processing Additionally, the presence of markers and the required setup can affect the naturalness of the subject's movement, potentially introducing biases into the analysis. These challenges significantly restrict the usefulness and accessibility of marker-based systems for routine clinical use and home monitoring.

Advances in sensors, signal processing and artificial intelligence (AI) have the potential to make quantitative gait analysis more accessible and scalable across both research and clinical settings. Wearables like inertial measurement units are portable and affordable, and provide spatiotemporal and angular gait parameters with correlation over 0.75 relative to marker-based optical MoCap. However, wearables face challenges like sensor drift, placement sensitivity, magnetic interference, and the need for calibration before every use, limiting their usability. Pressure-sensitive gait mats compute spatiotemporal gait parameters with high concurrent validity and intraclass coefficients of 0.92 to 0.99 when compared to marker-based MoCap or force plates. However, they lack joint angle measurements, portability, and are unsuitable for use outside laboratory environments.

Markerless MoCap systems, which include both single and multi-camera setups, leverage AI and computer vision for comprehensive gait analysis in practical settings without requiring body markers and specialized cameras, simplifying setup and saving time. Without markers, subjects can move more naturally, improving accuracy in real-world settings. These systems also avoid marker placement errors, enhancing repeatability across sessions. The automation of manual data processing further minimizes errors and streamlines workflow.

The use of digital images also increases accessibility and adaptability to various environments, as consumer-grade devices like smartphone cameras can be used.

Despite their advantages, markerless MoCap systems face limitations that hinder widespread clinical adoption Many rely on pretrained, open-source pose estimation models like OpenPose, which are not optimized for biomechanics applications. These models often mislabel or fail to label joint centers, and only compute two points per body segment, making them insufficient for calculating six degrees of freedom. Systems with biomechanics-focused models, such as Theia3D, offer greater accuracy but are expensive and require multi-camera setups (Kanko et al., 2021; Wren et al., 2023). Single-camera solutions, while cost-effective and easy to use, are less accurate than multi-camera setups. They often only estimate 2 dimensional (2D) instead of 3 dimensional (3D) poses, assuming movements occur strictly in the frontal or sagittal plane, making their accuracy heavily dependent on proper camera alignment. Occlusions and limited depth perception can further affect the precision of spatial parameters and joint kinematics parallel to the camera's line of sight. Other lower-cost systems target research or educational uses, do not automatically generate relevant clinical gait parameters, and may still require biomechanics expertise. To achieve broader clinical adoption, these systems must be integrated into more user-friendly platforms.

Embodiments of the present invention address these limitations by providing a 3DGait, an artificial intelligence (AI)-enhanced 3-Dimensional gait analysis system that operates with a single electronic device (e.g., consumer-grade camera), providing a streamlined, marker-less alternative to traditional systems. As discussed in greater detail later in the Specification, embodiments of the present invention integrate several machine learning algorithms to produce 49 angular, spatial, and temporal gait biomarkers, such as knee flexion, stride length, and double support time, commonly used in mobility analysis. When validated against a marker-based motion capture (MoCap) system (OptiTrack) using sixteen trials from eight healthy adult subjects performing a Timed Up and Go (TUG) test. Embodiments of the present invention achieved an overall average mean absolute error (MAE) of 2.27° and a Pearson's correlation coefficient (PCC) of 0.75 for angular biomarkers, with all angular biomarkers exhibiting an MAE under 5.5°. All spatiotemporal biomarkers from 3DGait showed errors under 15% relative to MoCap data. Temporal biomarkers (excluding TUG time) had errors under 0.1s. These results demonstrate 3DGait's accuracy and reliability, yielding performance comparable to traditional MoCap systems. 3DGait's accessible, non-invasive and single camera design makes it practical for use in non-specialist clinics and home settings, supporting patient monitoring and chronic disease management.

As such, embodiments of the present invention provide a more efficient, accurate, and robust lidar-based gait feature extraction and recognition using one or more artificial intelligence models to estimate points on an object to depict two dimensional and three dimensional poses of the object. In this manner, embodiments of the present invention provide an interdisciplinary approach that combines technological fields of computer vision, biomechanics, and artificial intelligence to better understand the potential and challenges of gait estimation in clinical care. Embodiments of the present invention can use lidar for plane estimation and scaling normalized three dimensional pose by identifying joint lengths and using short lidar for generating human body motion biomarkers as discussed in greater detail later in this Specification.

By performing the methodologies recited herein, embodiments of the present invention can enable healthcare professionals to capture high quality video recordings of a patient's gait, perform accurate and detailed analysis of gait parameters, provide visual representations and quantitative measures of gait analysis results, enhance the efficiency and effectiveness of gait analysis procedures which reduce the need for manual and subjective assessments. This improves diagnosis and treatment planning while complying with relevant device regulations, quality standards, and data protection requirements. Implementation of embodiments of the invention may take a variety of forms, and exemplary implementation details are discussed subsequently with reference to the Figures.

The invention will now be described in detail with reference to the Figures.

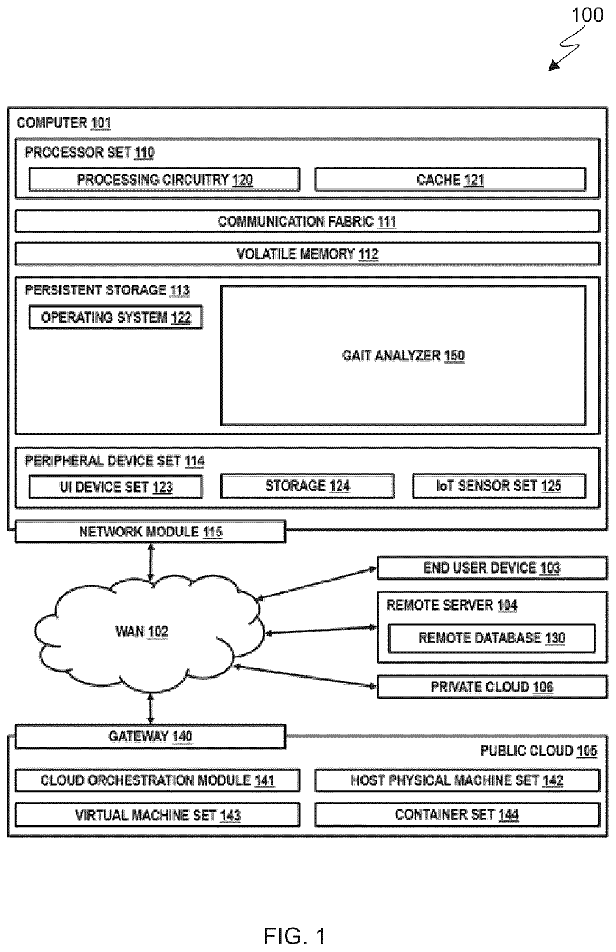

FIG. 1 depicts computing environment 100 illustrating components of computer 101 in accordance with an illustrative embodiment of the invention. It should be appreciated that FIG. 1 provides only an illustration of one implementation and does not imply any limitations with regard to the environments in which different embodiments may be implemented. Many modifications to the depicted environment may be made.

Various aspects of the disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, defragmentation, or garbage collection, but this does not render the storage device as transitory because the data is not transitory while it is stored.

Computing environment 100 contains an example of an environment for the execution of at least some of the computer code involved in performing the inventive methods, such as gait analyzer 150 (also referred to as program 150). In this embodiment, gait analyzer 150 can receive inputs from one or more sources (e.g., RGB video, depth images, etc.), analyze movements depicted in the received sources (e.g., two dimensional pose estimation, three-dimensional prose estimation, ground plane estimation, and two dimensional object detection), and estimate a gait associated with objects depicted in the received input. Pose estimation, as used herein refers to a computer vision task of determining the spatial positions and orientations of key body joints or key points in an image or video, typically used to analyze and understand the pose or movement of a person or object. In addition to program 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and program 150, as identified above), peripheral device set 114 (including user interface (UI), device set 123, storage 124, and Internet of Things (IOT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network, or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores.

Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip”. In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in program 150 in persistent storage 113.

Communication fabric 111 is the signal conduction paths that allow the various components of computer 101 to communicate with each other. Typically, this fabric is made of switches and electrically conductive paths, such as the switches and electrically conductive paths that make up busses, bridges, physical input / output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface type operating systems that employ a kernel. The code included in program 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images”. A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community, or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

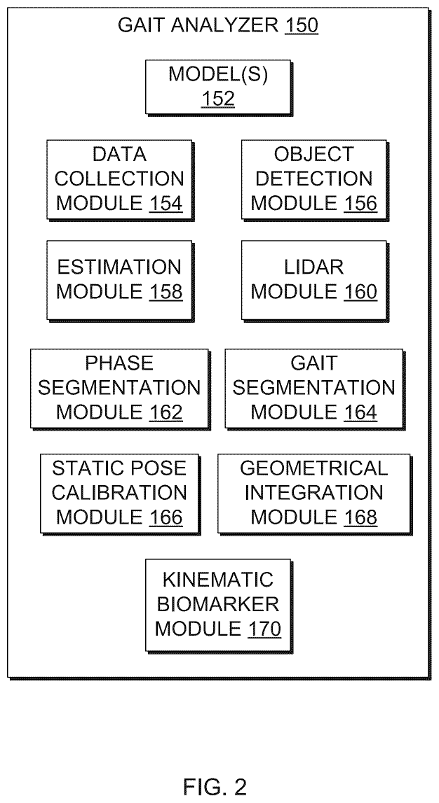

Gait analyzer 150 is a program, a subprogram of a larger program, an application, a plurality of applications, or mobile application software, which functions to analyze gait of an object using lidar based three dimensional gait measurements. In various embodiments, program 150 may implement the following steps: responsive to receiving information, identifying an object depicted in the received information; estimating points on the depicted object to depict two dimensional and three dimensional poses of the object; and generating measurements for biomechanical parameters that characterize movement of the object. In the depicted embodiment, program 150 is a standalone software program. In another embodiment, the functionality of program 150, or any combination programs thereof, may be integrated into a single software program. In some embodiments, program 150 may be located on separate computing devices (not depicted) but can still communicate over WAN 102. In various embodiments, client versions of program 150 resides on any other computing device (not depicted) within computing environment 100. In the depicted embodiment, program 150 includes model 152, data collection module 154, object detection module 156, estimation module 158, lidar module 160, phase segmentation module 162, gait segmentation module 164, static pose calibration module 166, geometrical integration module 168, and kinematic biomarker module 170 all shown and described in further detail with respect to FIG. 2. In this embodiment, gait analyzer 150 is can seamlessly authenticate, authorize, and manage users with precision.

Embodiments of the invention may contain various accessible data sources, such as database 130, that may include personal storage devices, data, content, or information the user wishes not to be processed. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Program 150 may provide informed consent, with notice of the collection of personal data, allowing the user to opt in or opt out of processing personal data. Consent can take several forms: opt-in consent imposes on the user to take an affirmative action before the personal data is processed, alternatively, opt-out consent imposes on the user to take an affirmative action to prevent the processing of personal data before the data is processed.

Program 150 enables the authorized and secure processing of user information, such as tracking information, as well as personal data, such as personally identifying information or sensitive personal information. Program 150 may provide information regarding the personal data and the nature (e.g., type, scope, purpose, duration, etc.) of the processing. Program 150 may provide the user with copies of stored personal data. Program 150 may allow the correction or completion of incorrect or incomplete personal data. Program 150 may allow the immediate deletion of personal data.

References in the specification to “one embodiment”, “an embodiment”, “an example embodiment”, etc., indicate that the embodiment described may include a particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether explicitly described.

FIG. 2 depicts a block diagram of a gait analyzer, in accordance with at least one embodiment of the present invention. In the depicted embodiment, program 150 includes model 152, data collection module 154, object detection module 156, estimation module 158, lidar module 160, phase segmentation module 162, gait segmentation module 164, static pose calibration module 166, geometrical integration module 168, and kinematic biomarker module 170.

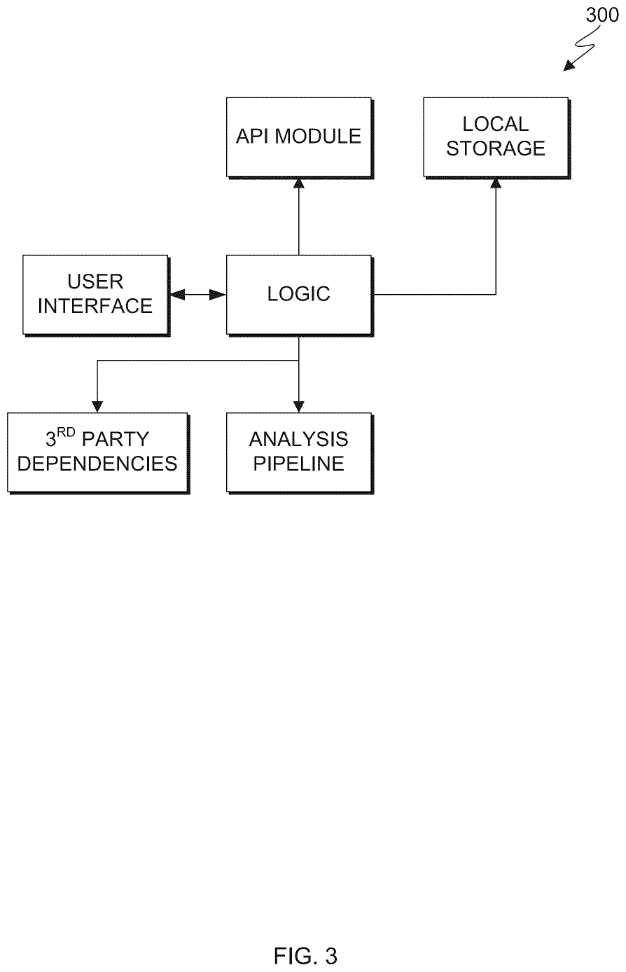

Model 152 is representative of a model utilizing deep learning techniques to train, calculate weights, ingest inputs, and output a plurality of solution vectors. In an embodiment, model 152 is comprised of any combination of deep learning model, technique, and algorithm (e.g., decision trees, Naive Bayes classification, support vector machines for classification problems, random forest for classification and regression, linear regression, least squares regression, logistic regression). In an embodiment, model 152 utilizes transferrable neural networks algorithms and models (e.g., long short-term memory (LSTM), deep stacking network (DSN), deep belief network (DBN), recurrent neural networks (RNN), compound hierarchical deep models, etc.) that can be trained with supervised, semi-supervised, or unsupervised methods. In the depicted embodiment, model 152 is a convolutional neural network (CNN) trained utilizing supervised training methods. The training of model 152 is depicted and described in further detail with respect to FIGS. 3-6.

In an example, for object detection and tracking, model 152 can be You Only Look Once (YOLO) (e.g., YOLOv7), which is a real-time object detection algorithm that processes an entire image in a single forward pass, predicting bounding boxes and class probabilities for multiple objects simultaneously. For 2D pose estimation, model 152 can be 1 pn model which is a deep neural network architecture designed for high-resolution visual tasks, particularly human pose estimation, and it utilizes a multi-resolution approach to capture fine-grained details. For 3D pose estimation, model 152 can be Videopose3D model which is simple and efficient AI model for 3D human pose estimation in video based on dilated temporal convolutions on 2D key points trajectories.

Selection for these models were based on accuracy for specific application settings, compatibility with operating system requirements and the need to have low latency to meeting processing goals of end user devices. In this embodiment, model 152 is optimized for training and validation loss. For example, Yolov7 model demonstrated a good accuracy with a faster processing time compared to other 2-stage detectors such as Faster-RCNN. For 2D pose estimation, gait analyzer 150 leverages the LPN network which is very lightweight and provides a similar accuracy compared to HRNet. It also showed better accuracy than other models such as Centrenet, YOLOv7 etc. Its bottom-up approach focuses on the pose key points on a single body and does not need a validity flag for the key points. For 2D to 3D pose estimation task, gait analyzer 150 utilizes VideoPose3D due to its proven efficacy and particular alignment with our project needs. VideoPose3D stands out for its ability to accurately interpret temporal dynamics, a crucial aspect when working with video data.

Data collection module 154 is capable of sending and receiving information from one or more components of computing environment 100. In this embodiment, data collection module 154 can receive information from one or more connected applications for audio and visual recording (e.g., RGB video) and lidar sensor data simultaneously during static pose calibration and Timed Up and Go (TUG) tests. For example, data collection module 154 can capture signals from both end user device cameras (e.g., such as tables, mobile telephones, etc.) and Lidar sensors. In this manner, data collect module 154 can create a comprehensive dataset that combines visual information with depth perception. This combined dataset is crucial for subsequent modules, enabling precise 3D pose estimation and generating valuable insights into gait patterns. The integration of visual and depth information enhances the overall accuracy and robustness of the system, ensuring a thorough understanding of human movement.

Object detection module 156 identifies one or more objects depicted in the received video. In this embodiment an object can be a person of interest. In other embodiments, an object can be corporeal being capable of movement. In instances where object detection module 156 identifies multiple individuals within the received video, object detection module 156 can automatically select a person of interest from the identified individuals for gait analyzer 150 to perform gait analysis on but maintains the ability to track each identified individual independently. In certain embodiments, object detection module 156 may include tracking capabilities. Subsequently, object detection module 156 can store tracking information for the identified person of interest.

In this embodiment, object detection module 156 detects a human subjects' boundaries including faces. Object detection module 156 utilizes the subject boundaries are for tracking, 2D pose estimation. For example, object detection module 156 utilizes the face boundaries for phase segmentation.

In this embodiment, object detection module 156 extract frames from the recorded bideo and utilizes 2D RGB images as input and outputs 2D bounding boxes [x, y, w, h] with [x, y] coordinates of the bounding boxes center and [w, h] dimensions of the bounding boxes for 2 classes (e.g., face and person-full body).

Object detection module 156 then assigns a tracking ID for each detected bounding box. If the bounding boxes correspond to the same one in the previous frame, object detection module 156 assigns the same ID is assigned, otherwise object detection module 156 assigns a new ID.

In some embodiments, object detection module 156 may utilize an algorithm as a post processing step to fill in any 2D object detection information for frames where the model did not provide an output. In this example, object detection module 156 utilizes the neighborhood frame 2D object detection information to estimate the bounding box boundaries in these missing image frames.

In this embodiment, object detection module 156 selects a person of interest with a rule-based method depending on the orientation of the camera (e.g., front view, 45 degree view or side view). Object detection module 156 then selects the bounding boxes corresponding of the person of interest and saves the bounding boxes with their corresponding detected face. Object detection module 156 can also save the camera index number associated with the extracted frames.

Estimation module 158 can leverage model 152 to estimate two and three dimensional pose of the selected object. In this embodiment, estimation module 158 can includes 2D pose estimation modules and 3D pose estimation models (not shown).

For 2D estimation, estimation module 158 detects certain key points on the human subject that are needed to find the biomarkers identified for gait analysis. In this embodiment, estimation module 158 can receive the bounding box for a person of interest from the frames of the recorded video. For example, object detection module 156 can identify the person region corresponding to the bounding box is extracted and resize to the input size of the model 256×192 and transmit the resized image is given to estimation module 158.

The module outputs (x, y) location of 28 key points for each frame individually. The object boundary of the person of interest is extracted and scaled according to the input requirements of a selected artificial intelligence model used. Estimation module 158 outputs a rescaled back to the image level. The AI model may also provide a confidence value for each key point. High confidence value indicates the reliability of the output location for that key point. Additionally, the module is responsible for generating normalized values for the 2D key points. This process ensures a comprehensive representation of the person of interest's movements and facilitates accurate 2D pose estimation based on the normalized key point values.

For 3D pose estimation, estimation module 158 takes the normalized input of the 2D key points that are provided by 2D pose estimation module. The (x, y) locations of each frame are normalized based on the requirements of the 3D pose estimation module used. The model outputs normalized 3d locations of these 28 key points usually between range of [−1,1]. The output is (x, y, z) for each key point.

For example, the design inputs for estimation module 158 performing 3D pose estimation include the normalized values for 2D key points per frame, obtained from the collected video through the 2D pose estimation model. These normalized values serve as crucial data for further processing, ensuring accurate and standardized representation of the key points throughout the video frames.

Estimation module 158 can then use an as a post processing step to fill in any 3D pose information for frames where module 152 did not provide an output. In this instance, estimation module 158 utilizes the neighborhood frame 3D pose information to estimate the pose in these missing image frames and outputs the extraction of 28 normalized 3D key points from the collected video. These 3D key points serve as essential data for the subsequent module, where biomarker computations are conducted. The accurate representation of these key points in three-dimensional space enhances the precision and depth of the subsequent analysis.

Geometrical integration module 168 plays a pivotal role in the comprehensive processing pipeline. This unit acts as the central hub where the outputs from various modules converge to create a cohesive and unified representation of the user's gait dynamics. It seamlessly integrates information derived from the phase segmentation module, incorporating temporal boundaries and key gait events. Additionally, it synergizes with the output of the 3D pose estimation module, aligning the spatial coordinates of key body points in a geometrically accurate manner. The unit employs sophisticated algorithms to reconcile data from disparate sources, ensuring consistency and accuracy in the final 3D reconstruction of the user's movements. By fusing temporal and spatial aspects, the Geometrical Integration Unit contributes to the generation of precise gait biomarkers, providing valuable insights for both clinicians and end-users in the realms of neurological and orthopedic care.

In this embodiment, geometrical integration module 168 performs smoothing, ground plane detection, scaling, and translation. For example, geometrical integration module 168 performs smoothing on the 3D pose estimation output module to address any potential jerkiness in the 3D output. Geometrical integration module 168 applies a biomechanics-consistent smoothing process enhancing the natural and realistic appearance of movements. In this manner, geometrical integration module 168 can smooth each individual joint using a custom filtering kernel depending on the jitteriness present.

For ground plane detection, geometrical integration module 168 utilizes LiDAR data to estimate the ground plane equation (a, b, c, d) of the floor where the subject is performing the Gait test on. Some filtering is performed to make the detection robust and efficient to noise that may be present in the scene.

Geometrical integration module 168 can scale 3D key point for each video frame based on subject-specific measurements which can be height or bone length of the person. It provides real-world coordinates for the 28 key points in 3D. and then outputs the translations that needs to be added to move the scaled 3D key points per frame which are currently in their own frame of reference with Pelvis at (0, 0, 0) to world (camera) coordinate system per frame.

For example, as inputs, geometrical integration module 168 receives the 28 normalized 2D & 3D key points from the collected video per frame (x, y) and (x, y, z).

Geometrical integration module 168 also takes in information from the LiDAR and the intrinsic parameter information from the RGB and LiDAR cameras. Additionally, geometrical integration module 168 intakes the subjects'height information.

As output, geometrical integration module 168 constructs a unified and coherent representation of the user's gait dynamics by harmonizing information from diverse modules, including the phase segmentation module and the 3D pose estimation module. In this manner, geometrical integration module 168 synthesizes a precise amalgamation of temporal and spatial data. The unit's output entails a geometrically accurate reconstruction of the user's movements in three dimensions along with ground plane estimation that is also used for kinematic analysis and biomarker computation.

Lidar module 160 maps the 3D estimated pose and scaled to real-world coordinates using geometrical information from Lidar sensor data and inferences made by model 152 as discussed in greater detail later in this Specification.

Phase segmentation module 162 employs real-time 2D key point estimation results from our pre-trained pose estimation model to generate a streamlined and interpretable phase segmentation for Time Up and Go (TUG) and 2-minute walk videos. Phase segmentation module 162 takes as input an RGB video of a TUG or walking test, along with parameters for frame rate and frame dimension. Phase segmentation module 162 extracts a set of meaningful and useful features from the body points and then perform smoothing on the signals. The smoothed features are used in a rule-based coarse-to-fine inference algorithm with consistency checking to enhance accuracy and robustness. In this way, phase segmentation module 162 effectively identifies temporal boundaries for various actions, including the start and end of sit-to-stand, walking, turning and stand-to-sit. Phase segmentation module 162 then transmits the phase information to subsequent modules for gait segmentation and biomarker computations.

In this embodiment, phase segmentation module 162 utilizes the 2D pose and 3D post outputs along with ground plane information to estimate the object's phase (i.e., walking, turning, sitting, sit-to stand, etc.), and gait estimation. For example, phase segmentation module 162 an receive as input, the 28 body 2D key points per frame, obtained from the collected video through the 2D pose estimation model, along with information on frame rate and frame dimension. This embodiment requires the camera to be set up at a 0-degree view angle with only a single subject in the field of view of the video. As outputs, phase segmentation module 162 can identify frame numbers representing the start and end of each TUG action. Phase segmentation module 162 can transmit the phase information to geometrical integration module 168 and kinetic biomarker module 164.

An example TUG output can include Total TUG time and 7 temporal boundaries between actions: 1) start of sit-to-stand, 2) start of walking, 3) start of turning, 4) start of walking back, 5) start of turning around, 6) start of stand-to-sit, 7) end of stand-to-sit.

An example two minute walk output can include temporal boundaries between walk and turn: [start_walk, start_turn₁, end_turn₁, start_turn₂, end_turn₂, . . . , start_turny, end_turn_{N}] for N number of turns. (for future updates).

Gait segmentation module 164 utilizes the normalized and scaled 3D coordinates of each key point per frame, as well as information on ground plane, walking segments and frame rate, to ascertain the commencement and conclusion of a valid gait cycle. It extracts data from the 3D body coordinates, specifically the shoulder and ankle key points, to form useful features.

For each walking segment, gait segmentation module 164 utilizes the left and right ankle signals are to estimate the start and end of a stride. With the ground plane parameters and foot coordinates, gait segmentation module 164 can compute the angles between the left/right foot and floor plane. This feature allows gait segmentation module 164 to determine the foot swing or heel strike positions, which subsequently identifies the temporal boundaries of stance and swing phases. Utilizing a rule-based inference algorithm and consistency checking, gait segmentation module 164 outputs timestamp for each gait cycle parameters, including left/right stride, step, single support and double support. The gait timestamps together with scaled 3D coordinates allow computation of stride/step length and speed for further analysis. The output of this gait segmentation module 164 also includes computation of count, mean and standard deviation of each gait parameters. This analysis contributes crucial insights into the dynamics of the user's gait, facilitating biomarkers computation for comprehensive understanding in clinical and research applications.

Examples as inputs include normalized and scaled 3D pose estimation results, and parameters of frame rate, ground plane equation (a, b, c, d) and walking segment boundaries (e.g., [start_walk₁, end_walk₁, start_walk₂, end_walk₂]). The 3D locations of key points are crucial for assessing the spatial dynamics of the subject's movements, offering detailed insights into the positioning of various body joints during the gait cycle. Concurrently, the ground plane information provides a contextual reference for these 3D locations, ensuring that the biomechanical computations are aligned with the real-world environment. This integrated input forms the foundation for the accurate and comprehensive assessment of biomechanical biomarkers within gait segmentation module 164.

In this way, gait segmentation module 164 generates informative outputs encompassing gait cycle frames, frame-specific details regarding left and right stride, step, single support and double support, along with the duration and speed taken for each discerned gait cycle. The count, mean and standard deviation of each gait parameters are also computed for statistical analysis in clinical applications.

Kinematic biomarker module 164 integrates essential components, beginning with the 3D output from the normalized 3D pose module. This normalized output serves as the basis for computing individual biomarkers that involve angular measurements (e.g., knee flexion, hip flexion). Utilizing a predefined mathematical framework and joint coordinates, kinematic biomarker module 164 accurately calculates these angles. Kinematic biomarker module 164 subtracts the joint angles from the static pose calibration module from these angles to normalize for inter-subject differences. If the static pose calibration module output is not available, this step will be skipped.

Kinematic biomarker module 164 utilizes the gait segmentation information to estimate the mean and standard deviation of movements within one gait cycle, constituting the final output. For spatial biomarkers like step length and stride length, outputs of gait segmentation module 164 aids in identifying the frames marking the commencement and conclusion of each step. Subsequently, the real-world 3D coordinates of body joints are used to determine the distance travelled, facilitating the computation of stride length and stride speed, among others.

Temporo-Spatial biomarkers, such as stride velocity and step velocity, leverage frame differentials and distance moved for their computation. The output encompasses mean, standard deviation, and min-max differentials, providing subject experts with comprehensive data for in-depth analysis. This multifaceted approach ensures a thorough assessment of the user's gait dynamics, contributing valuable insights for clinical and research applications.

In this way, kinematic biomarker module 164 leverages the 3D location of the 28 key points along with plane information and gait segmentation to generate various gait biomarkers, providing valuable insights for clinicians or end-users.

Example inputs kinematic biomarker module 164 can utilize come from static pose calibration module 166, phase segmentation module 162, gait segmentation module 164, the geometric integration module 168, and estimation module 158 (e.g., 3D pose estimation).

The normalized output from the preceding estimation module 158 (e.g., 3D pose estimation) serves as a foundational input. Kinematic biomarker module 164 utilizes phase segmentation information is used to determine the TUG time and direction of walking. Kinematic biomarker module 164 utilizes gait segmentation information for aligning biomarker computations with distinct gait cycle phases while geometrical integration module 168 contributes ground plane details and real-world 3D locations, enhancing the spatial accuracy of biomarker calculations.

Kinematic biomarker module 164 outputs a comprehensive set of biomechanical parameters derived from the input sources. These parameters include angular measurements such as knee flexion and hip flexion, computed using a predetermined mathematical and joint coordinates framework. For spatial biomarkers like step length and stride length, kinematic biomarker module 164 utilizes gait segmentation information to identify specific frames for the start and end of each step, leveraging the real-world 3D locations of body joints (key points) to estimate distances moved. Kinematic biomarker module 164 compute temporo-spatial biomarkers, such as stride velocity and step velocity, based on frame differentials and distances moved. Additionally, kinematic biomarker module 164 generates mean, and standard deviation providing valuable data for analysis by clinicians.

In this embodiment, gait analyzer 150 employs the following:
Algorithm Optimization: Choosing the most efficient algorithms and data structures that provide the best performance for the given context. Only using data that is needed for executing a certain function.
Loop Unrolling and Fusion: Reducing the overhead of loop control structures by unrolling loops (executing the loop body multiple times within a single loop iteration) or fusing adjacent loops to minimize iteration overhead.
Function Inlining: Replacing a function call with the actual code of the function, reducing the overhead associated with function calls.
Memory Access Optimization: Organizing data in memory to improve cache utilization and reduce disk access times.
Validating Inputs: Ensuring inputs are validated early to prevent unnecessary processing of invalid data.
Batch Processing: Where possible, designing inputs to be processed in batches reduced the overhead of processing individual items especially while using AI models.
Error Handling: The app should ensure errors are handled well, users are informed on the error that has occurred and steps have been taken to avoid unexpected crashes. The app should also log all actions, including user access, operation, and system errors for debugging, if needed, in future.
System Integration: The application seamlessly integrates with the backend system including API support for data retrieval and report generation.
Partial offline support: The application provides some critical functionalities like running gait analysis offline, with being able to sync some data when reconnected.
Compliance: The app should be designed and maintained properly so as to meet regulatory standards and successful security audits. This includes situations where all data must be deleted when the user uninstalls the app. The user data is stored securely on the cloud and robust synchronization techniques are implemented to avoid any async issues. The users should be able to use the app with minimal training and be generally positive about their experience while using the app.

In certain embodiments, gait analyzer 150 can be integrated with an Application Program Interface (API) application for backend processing for received information from a front end system (e.g., an application capable of sending recorded video). In this embodiment, the back end system communicates and pulls information from all the APIs in a 3d Gait application system (e.g., computing environment 100). The API layer is based on NodeJS as a framework because of its fast, efficient, and tight coupling between the client and the server and is highly scalable which allows the system to accommodate a wide range of customers.

FIG. 3 is a functional block diagram 300 of certain components of a computing environment for gait estimation using LIDAR-based 3d gait measurements, in accordance with at least one embodiment of the present invention. For example diagram 300 shows a high-level description of how components of computing environment 100 interacting with one another. In this example, the logic module may interact with the different components like API module, local storage, and analysis pipelines. The API module includes the session manager for logging in, logging out, authenticating users. The API module can get and update user data that are saved with the database manager. The analysis pipeline module may process the gait video. The 3rd-party dependencies module provides encapsulated solutions like math libraries for image processing. Finally, the UI module, shows how the user interacts with the various video capture applications.

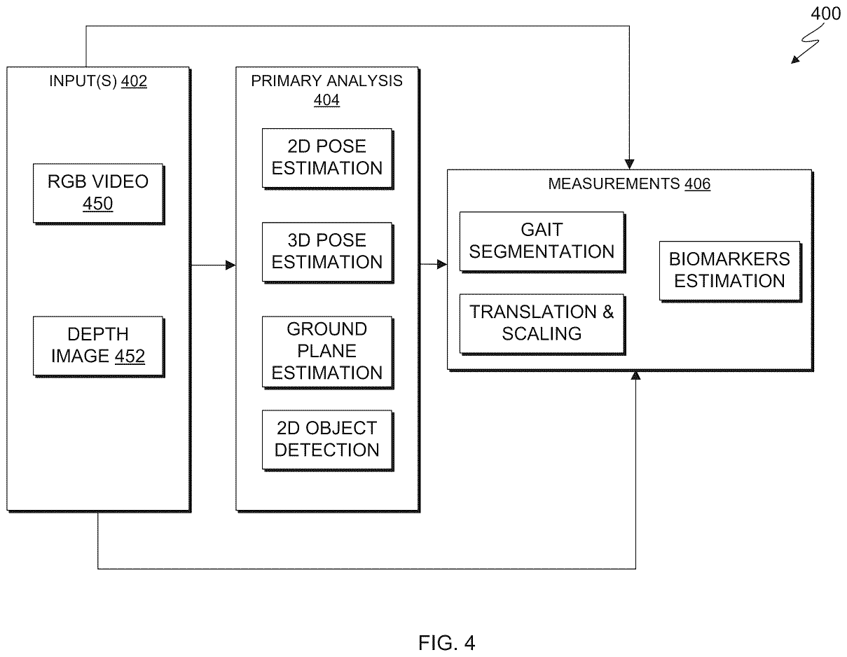

FIG. 4 depicts functional block diagram 400 illustrating an approach for gait estimation, in accordance with at least one embodiment of the present invention.

In this diagram, gait analyzer 150 can receive input(s) 402. In this embodiment, input(s) 402 can be one or more sources of media. For example, in certain embodiments, input(s) 402 can include RGB video 250 and depth image 252. RGB video 250 can represent an analog video signal based on red, blue, and green color models. In other embodiments, different types of videos can be used. Depth image 252 represents one or more images or image channels that contain information about the distance of surfaces of scene objects from a viewpoint. Each pixel in a depth image can represent the measurement of how far the scene and objects depicted in the scene is from the camera. Other inputs gait analyzer 150 can receive include raw depth data, camera intrinsic parameters, lidar camara intrinsic parameters, and gyroscopic data.

For example, an input can include a video of the subject performing a Timed Up and Go (TUG) test or a back-and-forth walk, Light Detection And Ranging (LiDAR) data and camera intrinsic parameters, which can be captured using an electronic device (e.g., a table with a camera). A tracking module (e.g., object detection module 156) can detect people in the video using an AI model based on the You Only Look Once (YOLO) system, pretrained on the Microsoft Common Objects in Context (MS-COCO) dataset. The tracking module then tracks unique individuals using a faster variant of deepSORT and identifies the Person of Interest (Pol) performing the task using a rule-based approach.

Gait analyzer 150 can perform primary analysis 404 on the received inputs. In this embodiment, gait analyzer 150 can perform 2D pose estimation (e.g., 28 2D key points (x, y)) and 3D pose estimation (e.g., 28 3D key points (x, y, z)), ground estimation (e.g., ground plane equation [a, b, c, d,]), and 2D object detection (e.g., bounding box coordinates [x, y, w, h]). In this embodiment, primary analysis 204 can be performed sequentially (e.g., outputs for 2D pose estimation are used as inputs for 3D pose estimation) while ground plane estimation can occur in parallel as it is independent of 2D and 3D pose estimation.

For example, estimation module 158 calculates the person of interest's 2D pose, the [x, y] coordinates of 28 key points on the human body which are adapted from the CAST marker set. Estimation module 158 uses the Lightweight Pose Network and includes post-processing steps to ensure spatial and temporal consistency. Estimation module 158 can then perform 3D estimation based on Videopose3D and transforms the 2D coordinates from the into 3D by analyzing the sequence of 2D movements across frames to infer depth and spatial relationships. The output 3D coordinates are scaled between −1.0 and 1.0, preserving the relative positions of the key points in 3D which are not yet translated into actual physical dimensions.

Next, a component of geometrical integration module 168 (e.g., a smoothing module reduces noise in the 3D pose using a 4th order 6 Hz low-pass Butterworth filter. Since the 3D coordinates are not yet in real-world dimensions, a scaling and translation module (another component of geometrical integration module 168) adjusts them using LiDAR data. First, it scales the normalized, smoothed 3D output to match the subject's physical body size based on LiDAR measurements. Then, it translates these poses to align with actual physical locations, using LiDAR or scene geometry. The final output is the [x, y, z] coordinates of each key point per frame within a real-world coordinate system, with the camera as the origin.

Phase segmentation module 162 identifies distinct phases of the TUG and walking tasks, such as sit, stand, walk, and turn, and outputs the frame numbers corresponding to the start of each phase. Phase segmentation module 162 analyzes the object detection and the 2D pose estimation outputs using an algorithmic approach. Gait analyzer 150 can utilize a ground plane estimation module (not shown) that leverages LiDAR data and the RANSAC algorithm to estimate the ground plane. Gait segmentation module 164 then uses the 3D coordinates of feet key points and the ground plane to determine the swing and stance phases, and gait cycle start and end points during the walking phases identified by the phase segmentation module 162.

Gait analyzer 150 can then generate measurements 406 by performing phase segmentation (e.g., Timed-Up-and-Go (TUG) segments [s, st, w, t, w, ts, s]). Gait analyzer 150 can then identify walking segments of the object and then performs gait segmentation analysis for that phase. Gait analyzer can then perform translation and scaling (e.g., 28 3D scaled key points (x, y, z)). Gait analyzer 150 can then perform biomarkers estimation (e.g., knee flexion, double support, time, step length, step duration, gait speed, etc.).

Finally, Kinematic biomarker module 170 calculates angular joint kinematics and spatiotemporal gait parameters based on the 3D pose, phase segments, and ground plane. Joint angles are computed based on the Joint Coordinate System framework and then averaged across gait cycles. Spatial parameters (e.g., step and stride length) are derived from foot movement, while temporal parameters (e.g., stride duration) are based on gait events. These results are presented in a user-friendly interface for easy analysis by clinicians or end-users.

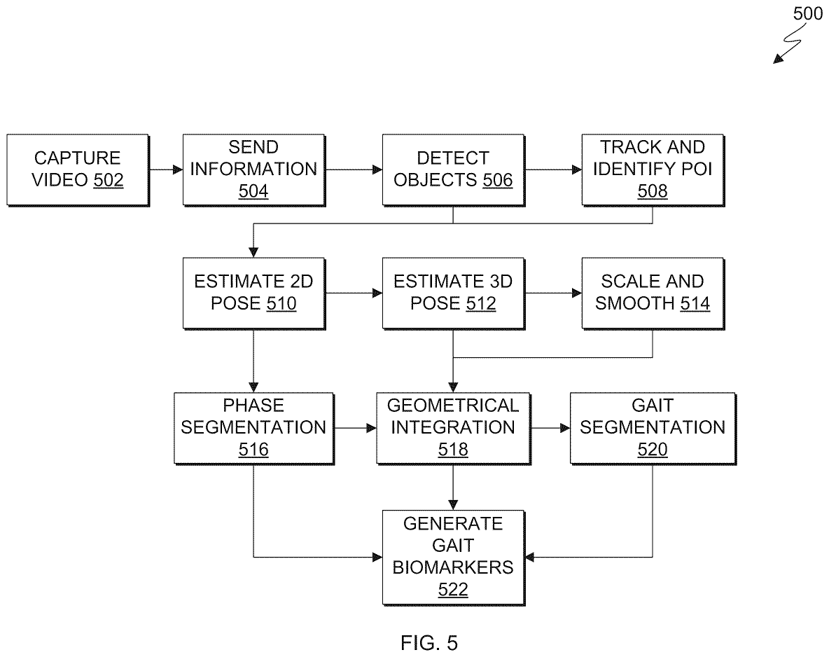

FIG. 5 is an example workflow 500 for generating gait biomarkers, in accordance with at least one embodiment of the present invention.

In this example, gait analyzer 150 collects or otherwise receives video using a single consumer-grade camera while a subject performs a Timed Up and Go (TUG) test that can range between three to eight meters. Embodiments of the present invention can also support four to ten meter walk test or a two min walk test in future releases. From the captured video, gait analyzer 150 can identify critical points in two dimensions (2D) on the object or person of interest depicted in the video and uses the output of 2D pose estimation to estimate the 3D pose of the person of interest. Gait analyzer 150 can then map and scale this 3D estimated pose into real world coordinates by extracting geometrical information available in lidar sensor data. Using this information along with phase and gait segmentation, gait analyzer 150 generates various gait biomarkers.

For example, in step 502 gait analyzer 150 captures video. In this embodiment, gait analyzer 150 can transmit instructions to an end user to capture and send video (e.g., step 504). In other embodiments, gait analyzer 150 can receive captured video from one or more end user devices.

Gait analyzer 150 can then leverage object detection module 156 to ingest 2D RGB images as input and output 2D bounding boxes (x, y, w, h) with (x, y) coordinates of the bounding boxes center and (w, h) dimensions of the bounding boxes for two classes-face and person (e.g., full body).

Gait analyzer 150 can then leverage a tracking module of object detection module 156 to ingest 2D RGB images as input as well as the bounding boxes detected and assigns a tracking ID for each bounding boxes detected (e.g., step 508). If the bounding boxes correspond to the same object seen in the previous frame, object detection module 156 assigns the same ID is assigned, otherwise object detection module 156 assigns a new ID. Once all the frames of the videos have been processed, object detection module 156 selects a person-of-interest (POI) with a rule-based method depending on the orientation of the camera (e.g., front view, 45 degree view or side view). Object detection module 156 then selects and saves the corresponding bounding boxes of the POI and with their detected faces.

Estimation module 158 can leverage a 2D pose estimation model to ingest the 2D RGB images as input as well as 2D person bounding boxes to estimate 2D pose (e.g., step 510). Estimation module 158 extracts a region of the person corresponding to the bounding box and resizes to the input size of the model 256×192. Estimation module 158 can then ingest the resized image and output the coordinates of the 28 key points.

In 2D to 3D pose estimation (e.g., step 512), a crucial step involves the meticulous handling of 2D per-frame input derived from the 2D pose component. This process begins with the alignment of frames in a sequential order, which is fundamental in preparing the input data for 3D model training. A key prerequisite for estimation module 158 utilizing a 3D model is the incorporation of a temporal window that spans multiple frames, as opposed to relying on single-frame data. This approach captures the dynamics of human motion more accurately. In this embodiment, estimation module 158 utilizes a window size of 243 frames. The specific window size is chosen to provide model 152 with an extensive temporal context, ensuring that each predicted 3D pose is informed by a comprehensive sequence of preceding movements. This depth of temporal data has a great positive effect on the precision of 3D pose estimations.

After the 3D pose estimation, the pelvis normalized 3D points are used to scale the 3D key points per frame (e.g., scale and smooth, step 514) by projecting lidar points 3D then to 2D images, leveraging the projected points to compute neck-sacrum lengths and scaling. Thereafter, gait analyzer 150 estimates the ground plane using the lidar data collected using the application. This is done using a RANSAC algorithm to ensure the ground plane is horizontal based on the input requirements of the end user device (e.g., tablet). Thereafter, the ground plane information, gait analyzer 150 uses the 3D location of the ankles to estimate location of person in 3D per frame. This location is analyzed sequentially and smoothing for jitter-free movement, The key points are then translated to this location.

Phase segmentation (e.g., step 516) takes 2D pose, video frames per second (fps), camera viewpoint (e.g., Cam1, Cam2, Cam 4) and frame resolution (e.g., width, height) as input. For example, phase segmentation module 162 an receive as input, the 28 body 2D key points per frame, obtained from the collected video through the 2D pose estimation model, along with information on frame rate and frame dimension and identifies frame numbers representing the start and end of each TUG action. Phase segmentation module 162 can transmit the phase information to geometrical integration module 168 and kinetic biomarker module 164.

Geometrical integration module 168 receives outputs from phase segmentation module 162 (e.g., step 516) and scaling and smooth (e.g., step 514) and incorporates temporal boundaries and key gait events (e.g., step 518).

Gait segmentation (e.g., 523) takes 3D pose, video fps, walking segments (start and end frames of walking phases) and ground plane parameters as input. utilizes the normalized and scaled 3D coordinates of each key point per frame, as well as information on ground plane, walking segments and frame rate, to ascertain the commencement and conclusion of a valid gait cycle computation of count, mean and standard deviation of each gait parameters.

In step 522, gait analyzer utilizes kinematic biomarker module 164 utilizes the 3D key point locations to calculate joint angles for each frame of the video. In this embodiment, JCS or relevant coordinate system is used for each biomarker.

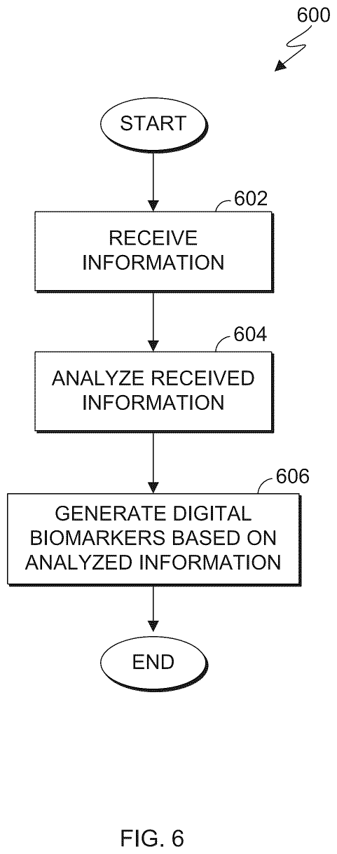

FIG. 6 depicts flowchart 500 depicting operational steps of a program on a computer within the computing environment of FIG. 1 for estimating measurements, in accordance with an embodiment of the present invention. By performing the method of flowchart 600, gait analyzer 150 can analyze gait of a moving object using lidar-based three dimensional gait measurements as described in greater detail below.

In step 602, gait analyzer 150 receives information. In this embodiment, information received can be one or more inputs from one or more sources of media (e.g., one or more RGB videos and one or more depth images). For example, a media source can depict a two dimensional image that can depict one or more objects in a physical space. In other embodiments, the media source can depict a three dimensional representation of an object in a virtual space. In other embodiments, gait analyzer 150 can utilize other sources of media.

For example, gait analyzer 150 can receive or otherwise collect information comprising color video and LiDAR data simultaneously using a tablet with a camera at 30 frames per second. In this example, the subject remained within the field of view of all tablets used for the duration of the trial. Although video was collected from multiple cameras for model training purposes, gait analyzer 150 uses data from one front facing camera (e.g., Tablet 1) as input. The remaining views were acquired for training purposes to enhance robustness of the AI models. Motion data was captured at the same time using a tracking system with 60 cameras at 120 Hz. Markers were placed according to the CAST marker set.

In this example, subjects performed a mix of 3.0 meter and 6.0 meter TUG tests, and a 6.0 meter back and forth walk. For the TUG test, subjects began seated on a stool, stood, walked 3.0 or 6.0 meters, turned, and returned to sit. Each subject completed 8 to 12 TUG trials per distance and twelve to sixteen trials of the 6 meter walk. The subjects did the same movement sequence twice-once with black body suit and reflective markers for capturing ground truth 3D data using Opti trak MoCap and once with regular clothing. While the bodysuit and markers are necessary for the MoCap system, they do not reflect typical clothing worn in real-world applications. Therefore, data was also collected in normal clothing to ensure our models perform well under realistic conditions.

In step 604, gait analyzer 150 analyzes the received information. In this embodiment, gait analyzer 150 analyzes the received information using one or more artificial intelligence algorithms to identify an object (e.g., a body) and detect points on the object depicted in the received information in 2D and 3D. Gait analyzer 150 can then overlay an image of the detected one or more points as a skeleton over the detected object.

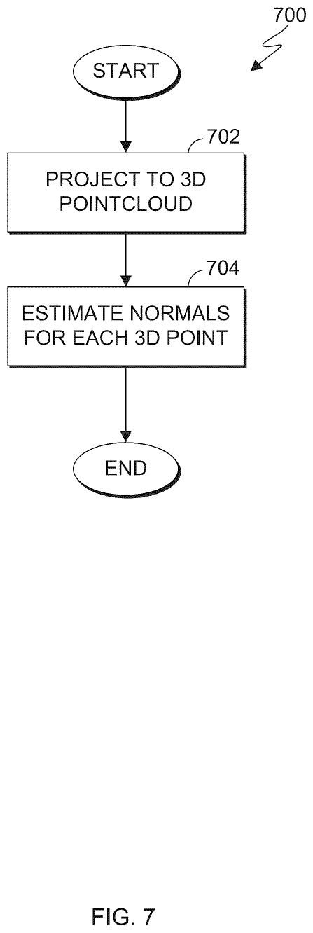

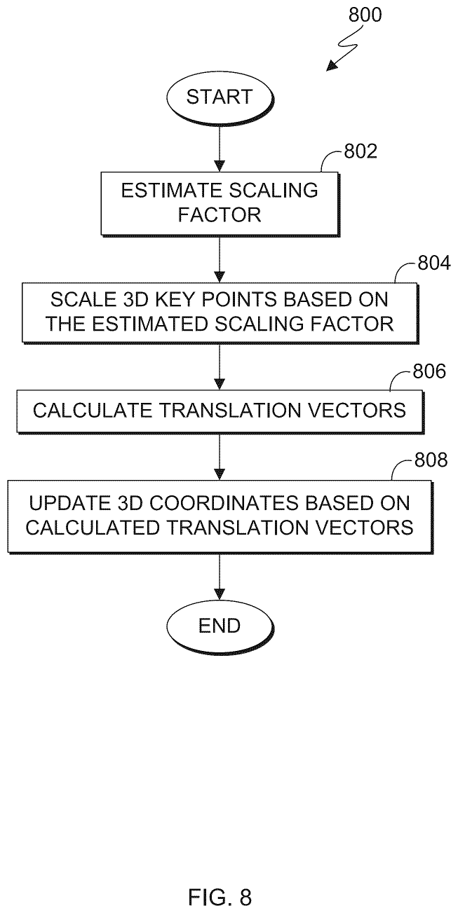

Gait analyzer 150 can then scale and transform the skeleton into a real world scale and its accompanying three dimensional coordinates. Gait analyzer 150 can scale and transform the skeleton by leveraging lidar to find the ground plane in real world coordinates. In this embodiment, gait analyzer 150 can estimate the ground plane for a depicted object by projecting to 3Dpoint Cloud for depth in defined range, estimating normal values for each point, using a random sample consensus (RANSAC) algorithm to find 3D points with normal oriented vertically, and extract ground plane equation and coefficients, as discussed in greater detail with respect to FIGS. 7 and 8.

In step 606, gait analyzer 150 generates digital biomarkers based on the analyzed information. As used herein, a biomarker refers to a measurable and quantifiable biological indicator that serves as a sign or characteristic of normal biological processes, pathogenic processes, or pharmacological responses to therapeutic interventions. In this embodiment, gait analyzer 150 generates multiple digital biomarkers to perform gait cycle estimation based on the scaling, translation, and ground plan information obtained using lidar data and custom artificial intelligence algorithms.

In some embodiments the multiple digital biomarkers such as gait speed can use lidar directly by finding the position of the human body when within range of lidar by recording time taken and distance moved to compute speed.

FIG. 7 depicts flowchart 700 depicting operational steps for ground plane estimation, in accordance with an embodiment of the present invention. By performing the steps of flowchart 700, gait analyzer 150 analyzes received information from step 602 of flowchart 600 to estimate three dimensional points of an object depicted from the received input.

In step 702, gait analyzer 150 projects to 3D point cloud. In this embodiment, gait analyzer 150 can ingest lidar data and intrinsic camera parameters to project detected points on the object to corresponding three dimensional points (e.g., x, y, z) in a three dimensional space. In this embodiment, gait analyzer 150 utilizes intrinsic camera parameters to ensure accuracy and for projection in the RGB camera's field of view.

In step 704, gait analyzer 150 estimates normal values for each 3D point. In this embodiment, gait analyzer 150 estimates normal values using normal coefficients [a, b, c] using a public library.

FIG. 8 depicts flowchart 800 depicting operational steps for scaling and translation, in accordance with an embodiment of the present invention. In this embodiment, gait analyzer 150 leverages the 3D pose estimation output resulting from flowchart 600 and scales the normalized key points to determine the actual height of the object depicted from the received input and translate to the actual position of the object.

In step 802, gait analyzer 150 estimate the scale factor (i.e., scaling coefficient) for the object. In this embodiment, gait analyzer 150 estimates the scaling factor by measuring the length between points on the object. Gait analyzer 150 select frames where the object is within a specified range and compute the average length between selected key points. In this embodiment, the object can be a person depicted within a received image, the specified range can be within one to fourteen meters, and the average length between selected key points can represent an average bone length of the person depicted in the image (e.g., length between neck and sacrum). In this embodiment, gait analyzer 150 can measure the length between the selected key points (e.g., bone/joint lengths) using lidar data in the selected frames, camera intrinsic parameters and lidar intrinsic parameters, and take the average to estimate a scaling coefficient.

For example, for identified walking phases one and two (e.g., outputs of phase segmentation module 162), gait analyzer 150 measures bone length by obtaining camera and lidar parameters, projecting regions around neck, sacrum, and toe 2D key points (e.g., pt) in depth/and lidar image to 3D (e.g., pt_3d).

Gait analyzer 150 then projects the 3D point (e.g., pt_3d) into an RGB image (e.g., pt_2d), finds the closest 2d key points (e.g., pt) and select their corresponding points from the 3D lidar point (e.g., pt_3d). In this manner, gait analyzer 150 can measure the distance between the neck and sacrum 3D points (i.e., bone length from neck to sacrum) and save the distance in a list and save position of the z position of the toe from the 3D point.

Gait analyzer 150 can then select data from the saved list when the z position from toe 3D point is between two to four meters and during walking phase one and two. Gait analyzer 150 can then interpolate data in the list if the selection has missing data using a cubic spline function and remove outlier values in the list when z-score is greater than two. Gait analyzer 150 can then return the average value from the list (e.g., the distance measure from lidar).

In step 804, gait analyzer 150 scales the 3D key points in a DataFrame based on subject-specific measures using lidar bone length measures. In this embodiment, gait analyzer 150 leverages the computed neck to sacrum distance from the 3D pose key points (e.g., output of 3D pose estimation), the estimated scaling coefficient (i.e., scale factor) with distance from lidar data and scale 3D key points with the scale factor.

In step 806, gait analyzer 150 calculates translation vectors for a sequence of frames along with selected foot information. In this embodiment, gait analyzer 150 calculates translation vectors for a sequence of frames by estimate plane equations corresponding to three centimeters above a ground level. For every frame of the sequence of frames, gait analyzer 150 determines which foot (e.g., left or right) is on the ground and computes the translation vector using plane coefficients, 2D points, scaled 3D points, and intrinsic camera parameters.

In step 808, gait analyzer 150 updates the 3d coordinates of key points by applying translation vectors. For example for every frame, gait analyzer 10 adds a translation vector to every key points.

Based on scaling, translation, and ground plane information obtained using lidar data, and artificial intelligence models, gait analyzer 150 can perform gait cycle estimation and output multiple digital biomarkers.

Biomarkers such as gait speed use lidar directly by finding the position of human body when within range of lidar by recording time taken and distance moved to compute speed.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A computer-implemented method for performing gait analysis using lidar based three dimensional measurements comprising:
responsive to receiving video information, identifying, by one or more processors of an object detection module, at least one object depicted in the received video information;
estimating points on the object, by one or more processors of an estimation module, to depict two dimensional and three dimensional poses of the object based on a threshold number of frames depicting the object; and
generating measurements, by one or more processors of a biomarker computation module, for biomechanical parameters that characterize movement of the object based on the estimated points on the object.

2. The computer-implemented method of claim 1, further comprising:
responsive to detecting more than one object depicted in the received video information, selecting, by the one or more processors of the object detection module, an object of interest based on camera orientation.

3. The computer-implemented method of claim 1, wherein estimating points on the object, by one or more processors of an estimation module, to depict two dimensional and three dimensional poses of the object based on a threshold number of frames depicting the object comprises:
generating, by one or more processors of an estimation module, bounding boxes corresponding to the object and a portion of the object with corresponding [x, y] coordinate values associated with a center of the bounding boxes and corresponding [w, h] dimensions of the bounding boxes for the object and the portion of the object.

4. The computer-implemented method of claim 3, further comprising:
resizing, by the one or more processors, a region corresponding to the generated bounded boxes of the object;
detecting, by the one or more processors, twenty eight, two dimensional key points of the object depicted within the bounding boxes for each frame depicted in the received video information; and
generating, by the one or more processors, normalized values for the detected twenty eight, two dimensional key points.

5. The computer-implemented method of claim 4, further comprising:
identifying, by one or more processors of a phase segmentation module, temporal boundaries for segments of movement based on the generated normalized values for the detected, twenty eight two dimensional key points and parameters for frame rate and frame dimension.

6. The computer-implemented method of claim 4, further comprising:
aligning, by the one or more processors of the estimation module, the normalized values for twenty eight two, dimensional key points to a threshold number of frames of a video in a sequential order;
estimating, by the one or more processors of the estimation module, three dimensional coordinate locations for the object based on neighboring frame three dimensional pose information in the threshold number of frames; and
generating, by the one or more processors of the estimation module, normalized three dimensional coordinate locations of the twenty eight, two dimensional key points.

7. The computer-implemented method of claim 6, further comprising:
mapping and scaling, by one or more processors of a geometrical integration module, the estimated three dimensional coordinate location into real world coordinates by extracting geometrical information available in lidar sensor data.

8. The computer-implemented method of claim 7, wherein extracting geometrical information available in lidar sensor data comprises:
smoothing, by the one or more processors, the normalized three dimensional locations of the twenty eight, two dimensional key points;
utilizing, by the one or more processors, lidar data to estimate ground plane equation of a surface that the object traverses;
scaling, by the one or more processors, three dimensional key points for each frame based on object specific measurements to provide real world coordinates for the normalized three dimensional locations of the twenty eight, two dimensional key points; and
performing translation on the scaled three dimensional key points for each frame to move corresponding three dimensional key points to a world coordinate system per frame.

9. The computer-implemented method of claim 8, further comprising:
identifying, by one or more processors of a gait segmentation module, frames marking commencement and conclusion of movement depicted by the object; and
estimating a mean and standard deviation for a distance traveled within one gait cycle.

10. The computer-implemented method of claim 1, wherein generating measurements for biomechanical parameters that characterize movement of the object comprises:
computing, by one or more processors of a kinematic and biomarker module, angular measurements for each frame depicted in the received video information.

11. A computer system for performing gait analysis using lidar based three dimensional measurements comprising:
a memory storing machine-executable instructions; and
a processor configured to access the memory storing the machine-executable instructions and execute the machine-executable instructions to:
responsive to receiving video information, identify at least one object depicted in the received video information;
estimate points on the object to depict two dimensional and three dimensional poses of the object based on a threshold number of frames depicting the object; and
generate measurements for biomechanical parameters that characterize movement of the object based on the estimated points on the object.

12. The computer system of claim 11, further comprising:
responsive to detecting more than one object depicted in the received video information, select an object of interest based on camera orientation.

13. The computer system of claim 11, wherein estimating points on the object, by one or more processors of an estimation module, to depict two dimensional and three dimensional poses of the object based on a threshold number of frames depicting the object comprises:
generate bounding boxes corresponding to the object and a portion of the object with corresponding [x, y] coordinate values associated with a center of the bounding boxes and corresponding [w, h] dimensions of the bounding boxes for the object and the portion of the object.

14. The computer system of claim 13, further comprising:
resize a region corresponding to the generated bounded box of the object;
detect twenty eight two dimensional key points of the object depicted within the bounding boxes for each frame depicted in the received video information; and
generate normalized values for the detected twenty eight, two dimensional key points.

15. The computer system of claim 14, further comprising:
identify temporal boundaries for segments of movement based on the generated normalized values for the detected twenty eight, two dimensional key points and parameters for frame rate and frame dimension.

16. The computer system of claim 14, further comprising:
align the normalized values twenty eight, two dimensional key points to a threshold number of frames of a video in a sequential order;
estimate three dimensional coordinate locations for the object based on neighboring frame three dimensional pose information in the threshold number of frames;
and
generate normalized three dimensional locations of the twenty eight two dimensional key points.

17. The computer system of claim 16, further comprising:
map and scale the estimated three dimensional coordinate locations into real world coordinates by extracting geometrical information available in lidar sensor data.

18. The computer system of claim 17, wherein extracting geometrical information available in lidar sensor data comprises:
smooth the normalized three dimensional locations of the twenty eight, two dimensional key points;
utilize lidar data to estimate ground plane equation of a surface that the object traverses;
scale three dimensional key points for each frame based on object specific measurements to provide real world coordinates for the normalized three dimensional locations of the twenty eight, two dimensional key points; and
perform translation on the scaled three dimensional key points per frame to move corresponding three dimensional key points to a world coordinate system per frame.

19. A computer program product for performing gait analysis using lidar based three dimensional measurements comprising:
one or more computer readable storage media having computer-readable program instructions stored on the one or more computer readable storage media, said computer-readable program instructions, when executed by one or more processors stored on the one or more computer readable storage media, cause the one or more processors to:
responsive to receiving video information, identify at least one object depicted in the received video information;
estimate points on the object to depict two dimensional and three dimensional poses of the object based on a threshold number of frames depicting the object; and
generate measurements for biomechanical parameters that characterize movement of the object based on the estimated points on the object.

20. The computer program product of claim 19, wherein the one or more computer readable storage media further comprise computer-readable program instructions to:
responsive to detecting more than one object depicted in the received video information, select an object of interest based on camera orientation.
